(19) United States
(12) Patent Application Publication
(10) Pub. No.: US 2024/0415394 A1
(43) Pub. Date: Dec. 19, 2024

(54) DEVICE FOR COLLECTING FLUID LOSSES AND SYSTEM FOR QUANTIFYING FLUID LOSSES THAT COMPRISES SUCH A DEVICE

(71) Applicant: TAM TELESANTE, SALON DE PROVENCE (FR)

(72) Inventors: Séverin SLOVE, SALON DE PROVENCE (FR); Pierre RAYNAL, SALON DE PROVENCE (FR)

(21) Appl. No.: 18/727,040

(22) PCT Filed: Apr. 14, 2023
(86) PCT No.: PCT/FR2023/050538

§ 371 Date: Jul. 5, 2024

(30) Foreign Application Priority Data
Apr. 14, 2022 (FR) FR2203470

(51) Int. Cl. A61B 5/02; A61B 5/15

(52) U.S. Cl. CPC A61B 5/02042; A61B 5/150045; A61B 5/150366

(57) ABSTRACT

The invention relates to a device for collecting fluid (10) including a tubular body (11) having a first open free end (13) and a second open free end (14), and including at least one conduit (30) and a flared section (20) extending from the conduit (30), and having an increasing diameter from the conduit (30) to the first open free end (13), and a part (21), referred to as top part, of the first open free end (13) being more radially spaced relative to the conduit (30) than another part (22), referred to as bottom part, of the first open free end (13), which is diametrically opposite the top part (21). It also relates to a system for quantifying fluid loss including such a device and a quantification receptacle connected to the conduit (30) of the device (10).

The invention relates to a device for collecting fluid loss and a system for quantifying fluid loss including such a device.

Childbirth naturally induces blood loss for the patient who has given birth.

However, if the blood loss is at least 500 mL within twenty-four hours postpartum, this blood loss is then referred to as Postpartum Haemorrhage (known as “PPH”).

PPH affects approximately 10% of births.

Among cases of PPH, a case of PPH is referred to as “severe” if the blood loss is at least 1000 mL within twenty-four hours postpartum. Severe PPH thus affects approximately 1% to 2% of births.

In recent years, PPH has represented the main cause of pregnancy-related maternal mortality in France, and even worldwide.

A report by the French National Committee of Experts on Maternal Mortality from 2007 to 2009, published in 2013, reveals a maternal mortality rate of 9.6 per 100,000 live births. Among the obstetrical causes of death, 18% are associated with PPH.

The main known risk factors of haemorrhage do not allow selection with sufficient sensitivity and specificity of the patients who need specific preventive measures before giving birth. The majority of cases of PPH occur without any risk factor being clearly identified. Hence, it is not possible based on current knowledge to recommend a PPH prevention strategy based on the identification of risk factors for a given patient, before giving birth.

The first hours postpartum (typically between two and six hours) are therefore crucial in PPH monitoring.

At the present time, for a vaginal delivery, postpartum blood loss is evaluated using a graduated collection bag which is placed under the patient's buttocks. The bag is left as such as long as the patient is in the gynaecological position, i.e. approximately 20 to 30 minutes postpartum, then it is removed when the patient is recumbent. Monitoring of the patient in the delivery room is continued for up to two hours postpartum, but the exact quantification of the blood loss then becomes impossible in the recumbent position, because in this position, if the collection bag is left in place, blood flows into the patient's back and/or various protections, undersheets and/or sheets. Quantification with a collection bag is currently considered to induce a 30% underestimation of blood loss.

For a caesarean, perioperative blood loss is quantified by the volume of liquid collected in suction jars. However, this collected volume only partially reflects the blood lost during the perioperative period, in particular because a substantial portion of the aspirated fluid may be amniotic fluid, and a non-negligible portion of the blood flows through the patient's vagina, and along the surgical fields. After caesarean delivery, the patient is monitored for approximately two hours in a recovery room (RR), but no means for quantifying blood loss is then set up. Only the blood flowing into the sheets may alert the monitoring team if the quantity appears to be too substantial.

Thus, in any case, there is only an estimation of the quantity of blood lost over the crucial postpartum time period, and no quantification is carried out whereas precision in the measurement of the blood volume lost is key information for diagnosing PPH.

In the report mentioned above, approximately 74% of PPH-related maternal deaths had a diagnostic failure on account of two essential elements: lack of blood loss quantification, both for vaginal and caesarean births, and monitoring failure in respect of the patient's clinical parameters, including the exact blood loss volume.

This lack of monitoring and exact knowledge of the blood volume lost by the patient results in a lack of awareness of the situation among delivery room professionals.

Yet, failure to understand that the patient has PPH is dangerous; PPH may quickly deteriorate and result in substantial morbidity (surgical procedures; hospitalisation in intensive care unit, etc.), or even death. Therefore, time is lost for establishing the diagnosis and there is a loss of chance of the patient's care, or even of her survival.

Many deaths caused by PPH could be prevented if blood loss could be better quantified.

Indeed, these haemorrhages represent the greatest proportion of preventable deaths, namely approximately 83.9% according to an INSERM study.

Therefore, there is a need for equipment for quantifying the blood loss of patients who have given birth, for both vaginal and caesarean births.

To this end, a device for collecting fluid loss is proposed, including: a tubular body having a first open free end and a second open free end, the tubular body including at least:
a conduit, including the second open free end, and
a flared section, extending from the conduit and including the first open free end, the flared section having an increasing diameter from the conduit to the first open free end, and
   a so-called top part of the first open free end being more radially spaced relative to the conduit than another so-called bottom part of the first open free end, which is diametrically opposite the top part.

The flared section is thus configured to be inserted, in particular in a patient's vagina postpartum, for example in the vicinity of the outer orifice of the cervix.

The flared section thus forms a collection cup which is asymmetrical and is configured to collect a fluid flow.

At least the flared section, forming the collection cup, is for example made of a relatively flexible and deformable material to be introduced easily, for example into a vagina.

The material includes for example an elastomer.

It consists for example of a biocompatible material.

The material has for example a hardness between 40 shore A and 80 shore A.

The first end thus forms for example a flow inlet end in the device, and the second open free end thus forms an outlet end of the flow from the device.

The asymmetric shape of the flared section improves the fluid collection and contributes to a satisfactory flow of the flow through the device to discharge the fluid loss.

This shape also makes it possible to limit a siphon effect capable of biasing a quantification of the loss.

The conduit refers here to a tube, of regular cross-section, in particular of circular or oval cross-section.

The conduit has for example a substantially decreasing diameter from the flared section to the second open free end, which helps facilitate the fluid flow.

The asymmetric part of the flared section has a shape such that the conduit is located at a lowest level, when the device is fitted in a patient who is generally recumbent on her back, making it possible to reduce the step or siphon effect. Thus, any flow engaged in the flared part flows into the conduit with a minimal dead volume.

Such a device for collecting fluid loss is thus particularly configured to form a vaginal device for collecting blood loss.

For example, for the patient lying on her back, the top part is configured to be positioned in the patient's vagina towards the bladder whereas the bottom part is configured to be positioned towards the rectum.

For example, the flared section includes a first side, referred to as top side, which includes the top part of the first open free end.

For example, the flared section includes a second side, referred to as bottom side, which includes the bottom part of the first open free end.

For example, the top side of the flared section is then flared relative to the conduit according to a greater average angle than the bottom side of the flared section.

For example, the flared section includes a lateral side, referred to as left lateral side, and another lateral side, referred to a right lateral side, which is diametrically opposite the left lateral side; and the left lateral side and the right lateral side are flared relative to the conduit according to the same average angle.

For example, a plane passing through the top side and the bottom side of the flared section forms a plane of symmetry, when the device is in a free state.

The flared section has for example a general oblique truncated cone shape, of which a base is formed by the first open free end.

For example, the first open free end has an oval or circular cross-section.

For example, the conduit then extends from a vertex of the oblique truncated cone formed by the flared section.

For example, a centre of the first open free end is off-centred relative to a centre of the conduit, for example relative to a section of the conduit whereby the flared section is connected to the conduit.

The centre denotes for example the geometric centre.

For example, the bottom side of the flared section has a substantially straight profile.

For example, the bottom side of the flared section is then aligned relative to the conduit.

For example, a bottom side of the device forms a straight line.

The bottom side of the device includes here the bottom side of the flared section and a bottom side of the conduit located in an extension of the bottom side of the flared section.

For example, a plane passing through the top side and the bottom side of the device forms a plane of symmetry, when the device is in a free state.

For example, the top side of the flared section has a substantially “S”-shaped profile.

Such an at least partially “S”-shaped shape appeared to provide the device with better ergonomics. In particular, the device holds more readily in place in the vagina while producing little, or no, discomfort for the patient.

For example, the collection device includes an insertion holding system.

Such an insertion holding system is configured to prevent the device from slipping out of the zone wherein it is introduced, in particular out of the vagina, and thus helps hold the flared section in position, for example in the vagina.

The insertion holding system includes for example a bead formed at the outer periphery of the first open free end.

Such a bead furthermore provides a rounded shape to an edge of the first open free end, which also facilitates an insertion of the device, in particular into the vagina, as well as its removal.

Such a bead helps preserve the desired shape of the first open free end, that is wide open, in particular when the flared section is in the vagina and may thus be subjected to stress which could deform it.

In an example embodiment, the collection device includes a positioning indicator.

Such a positioning indicator makes it possible to check how the device is positioned and oriented.

In an example embodiment, the positioning indicator includes a rib which extends longitudinally along a part of the top side of the device.

Such a rib makes it possible to ensure the alignment of the device when it is in place, for example in the patient's vagina, and to monitor that it remains thus, for example along a sagittal plane.

Such a rib is for example also possibly configured to rigidify the collection device in flexion.

The top side of the device, opposite the bottom side of the device, includes here a top side of the conduit and the top side of the flared section.

The rib extends for example longitudinally along at least a part of the top side of the conduit, or also along a part of the top side of the flared section.

The collection device being for example of round or oval cross-section, the top side then denotes a line extending longitudinally from the top side of the first open free end which has a maximum spacing relative to the conduit. Said line then forms a top line of the device.

Such a rib may also be configured to limit a rotatability of the collection device, in particular when it is in place in the vagina, and thus help hold the desired orientation of the device once in place.

In an example embodiment, the conduit, which extends the flared section by thus forming a tube, includes a connection endpiece configured to connect a quantification receptacle thereto, such as for example a graduated collection bag.

For example, the connection endpiece includes the second open free end.

The connection endpiece includes for example a cannula.

The conduit then also includes, by definition, a flow section, disposed between the flared section and the connection endpiece.

According to another aspect of the invention, a system for quantifying fluid loss is also proposed, including a device for collecting fluid loss as described above and a quantification receptacle connected to the conduit of the collection device.

Such a quantification system thus makes it possible to obtain objective and accurate information of the fluid volume, in particular outward blood loss in the first hours postpartum.

The invention, according to one example embodiment, will be better understood and its advantages will become more apparent upon reading the following detailed description, given by way of example and in no way limiting, with reference to the appended drawings wherein:

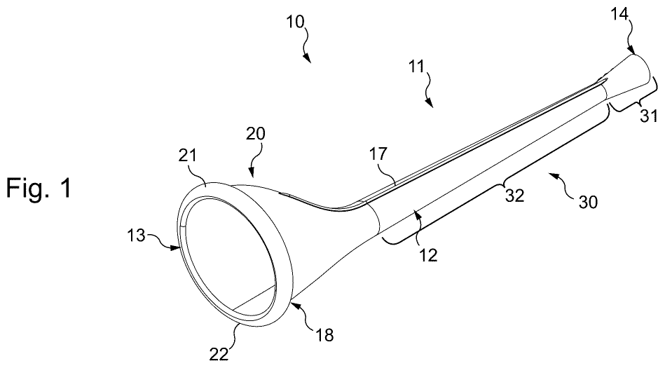

FIG. 1 illustrates a perspective view of a device for collecting fluid loss according to an example embodiment of the invention;

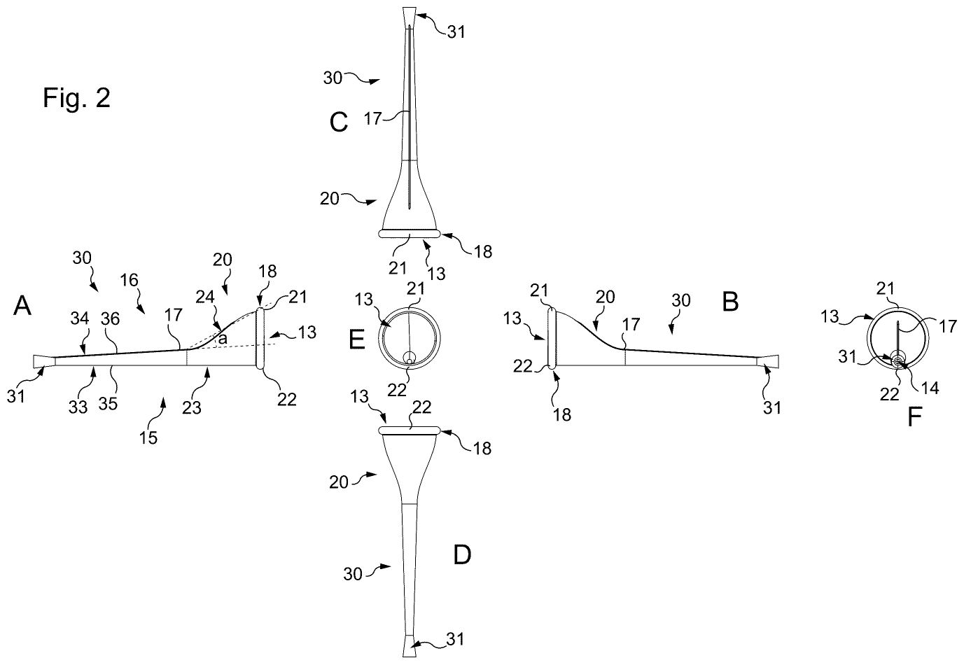

FIG. 2 represents the device of FIG. 1, respectively left and right profiles (FIGS. 2A and 2B), a top view (FIG. 2C), a bottom view (FIG. 2D), a front view (FIG. 2E) and a rear view (FIG. 2F); and

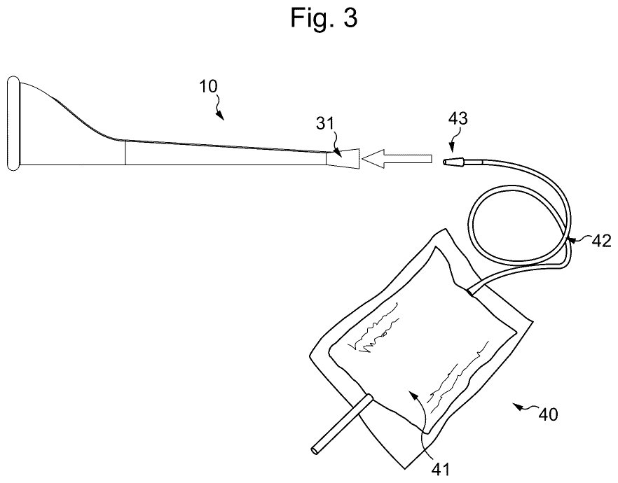

FIG. 3 represents the device of FIGS. 1 and 2 with a quantification receptacle to be connected thereto.

Identical elements shown in the aforementioned figures bear identical reference numbers.

FIG. 1 shows a device for collecting fluid loss 10 according to an example embodiment of the invention.

The collection device 10 includes a tubular body 11.

The tubular body 11 essentially consists of an enclosure wall 12 with variable cross-sections.

The tubular body 11 includes two open free ends, between which the enclosure wall 12 extends.

The two open free ends include a first open free end 13 and a second open free end 14.

As shown in FIG. 1, the tubular body 11 includes two major sections:
a flared section 20 which includes the first open free end 13, and
a conduit 30, including here the second open free end 14, and which extends from the flared section 20.

The flared section 20 is configured to be inserted, in particular, into a vagina, in particular of a postpartum patient.

The conduit 30, which extends the flared section 20, is configured to guide a flow, preferably to a receptacle connected to the conduit 30 for this purpose.

The first open free end 13 forms for example a flow inlet end and the second open free end 14 forms a flow outlet end.

Thus, according to a preferred option, the conduit 30 includes a connection endpiece 31.

The connection endpiece 31 is therefore configured to connect a quantification receptacle thereto, such as for example a graduated collection bag, as illustrated in FIG. 3.

The connection endpiece 31 then includes here the second open free end 14.

The connection endpiece 31 includes for example a cannula.

The conduit 30 then includes, by definition, a section referred to as flow section 32, disposed between the flared section 20 and the connection endpiece 31.

In the present example embodiment, the conduit 30 has a circular cross-section.

Furthermore, the flow section 32 has here a substantially decreasing diameter from the flared section 20, at least to the connection endpiece 31, which helps facilitate the fluid flow.

In the present example embodiment, the connection endpiece 31 has, on the other hand, a substantially increasing cross-section from the flow section, and here to the second open free end 14, in order to facilitate the connection of the quantification receptacle.

Moreover, according to an advantageous feature of the invention, the flared section 20 has an increasing diameter from the conduit 30 to the first open free end 13.

In the present example, the flared section 20 also has a circular cross-section; it could however be oval or otherwise, as needed.

As shown in FIG. 1, the flared section 20 extends however asymmetrically relative to the conduit 30.

In particular, a part, referred to as top part 21 of the first open free end 13 is more radially spaced relative to the conduit 30 than another part, referred to as bottom part 22 of the first open free end 13, which is diametrically opposite the top part 21.

The flared section thus forms a collection cup, in particular intra-vaginal, which is asymmetrical and is configured to collect a flow of fluid, in particular of blood loss.

The flared section 20 has for example a general oblique truncated cone shape, of which a base is formed by the first open free end 13, and a vertex of which is extended by the conduit 30.

A geometric centre of the first open free end 13 is then off-centred relative to a geometric centre of the conduit 30.

The asymmetric shape of the flared section 20 is for example more visible in FIGS. 2A and 2B which shows a profile view of the collection device 10.

For example, the collection device 10 includes a bottom side 15 and a top side 16 which are diametrically opposed.

The collection device 10 having a circular cross-section, a side denotes here a line extending longitudinally from one end to the other of the tubular body 11.

Thus for example, the top side 16 represents the maximum height line of the device 10 relative to the bottom side 15.

The bottom side 15 of the device is therefore here composed of the bottom side 23 of the flared section 20 and a bottom side 33 of the conduit 30 (including a bottom side 35 of the flow section 32), and the top side 16 of the device is then composed of a top side 24 of the flared section 20 and a top side 34 of the conduit 30 (including a top side 36 of the flow section 32).

An angle “a” (illustrated in FIG. 2A) formed by the top side 24 relative to the conduit 30 (in particular relative to the flow section 32 of the conduit) is considered as positive, between 0° and 900 for example, preferably between 0° and 45°.

Similarly, an angle “b” (not illustrated) formed by the bottom side 23 relative to the conduit 30 is considered as positive, between 0° and 90° for example, preferably between 0° and 45°.

Furthermore here, the top side 24 of the flared section 20 includes the top part 21 of the first open free end 13, and the bottom side 23 of the flared section 20 includes the bottom part 22 of the first open free end 13.

The top side 24 is then flared relative to the conduit 30 according to an average angle “a” greater than the average angle “b” of the bottom side 23.

In particular, the top part 21 of the first open free end 13 is here the most off-centred part of the first open free end relative to the conduit 30, i.e. having a maximum radial offset (therefore corresponding to a maximum average angle “a”).

In the example embodiment represented, the top side 24 of the flared section 20 has a substantially “S”-shaped profile.

The top side 24 of the flared section 20 and the top side 36 of the flow section 32 then form here a curved line, which is “S”-shaped.

Furthermore, the bottom part 22 of the first open free end 13 is here the part of the first open free end which is the least off-centred relative to the conduit 30, i.e. having a minimum radial offset.

In the embodiment shown here, this minimum radial offset is even zero.

The angle “b” formed by the bottom side 23 relative to the conduit 30 is then considered as zero.

The bottom side 23 of the flared section 20 has furthermore a substantially straight profile.

The bottom side 23 of the flared section 20 and the bottom side 35 of the flow section 32 furthermore form here a straight line.

Furthermore, the top side 36 of the flow section 32 here has an increasing height from the connection endpiece 31 relative to the bottom side 35, corresponding to the increasing diameter.

However, this diameter, and therefore the height, could be constant.

FIGS. 2C and 2D show the collection device 10 in a top and bottom view respectively.

These figures show that the device has a symmetry relative to a median plane (not shown), which corresponds for example to a sagittal plane of the patient when the device is in place.

Such a median plane then passes through the top side 16 and the bottom side 15 of the device 10.

Laterally, the flared section 20 therefore has a symmetric flared shape.

A right lateral part of the of the first open free end 13 is then just as radially spaced relative to the conduit 30 as a left lateral part of the first open free end 13, which is diametrically opposite the right lateral part.

Moreover, as illustrated by FIGS. 1 and 2C in particular, the collection device 10 includes a positioning indicator 17.

The positioning indicator 17 makes it possible to check the desired positioning of the device.

In the present example embodiment, the positioning indicator 17 is formed by a rib.

Such a rib is thus configured to limit a rotatability of the collection device 10 once it has been positioned.

It also makes it possible to see if the device is not twisted.

In particular, in this example, the collection device 10 is configured so that the top side 16 is positioned in the vagina towards the bladder, i.e. upwards when the patient is recumbent on her back.

The rib extends here along a part of a top side 16 of the device 10.

The rib is therefore here in the plane of symmetry of the device 10.

In particular, the rib extends here along a part of the top side 34 of the conduit 30 and along a part of the top side 24 of the flared section 20.

In particular, the rib extends here all along the top side 36 of the flow section 32.

It thus covers a connection zone between the conduit 30 and the flared section 20, which helps hold the shape of the device 10 when it is in place.

Moreover, as illustrated by FIGS. 1 and 2, the collection device 10 includes an insertion holding system 18.

Such an insertion holding system 18 is configured to prevent the device 10 from slipping out of its insertion zone and thus helps hold the flared section 20 in position in said zone.

The insertion holding system 18 here consists of a bead formed at the outer periphery of the first open free end 13.

Such a bead furthermore provides a rounded shape to the edge of the first open free end 13 of the device 10, which also facilitates an insertion of the device into the vagina as well as its removal.

Such a bead also helps preserve the open shape of the first open free end 13, in particular when the flared section 20 is in place, for example in the vagina.

FIG. 3 shows an example of a system for quantifying fluid loss 100 including a device for collecting fluid loss 10 as shown in FIGS. 1 and 2, and a quantification receptacle 40, ready to be connected to the conduit 30 of the device 10.

The quantification receptacle 40 includes for example here essentially a graduated collection bag 41, and a tube 42. The tube 42, on one hand, opens into the bag 41 and, on the other, is ended with an endpiece 43 configured to be connected to the cannula 31 of the conduit 30 of the device 10, for example by being inserted therein.

The endpiece 43 is for example a conical coupling.

Thus, if the device 10 is in place in a patient's vagina, the conduit makes it possible to connect a bag for collecting and quantifying the fluid loss, in particular postpartum blood loss, thus helping monitor postpartum haemorrhages.

1. Device for collecting fluid loss (10) including:
a tubular body (11) having a first open free end (13) and a second open free end (14), the tubular body (11) including at least:
a conduit (30), including the second open free end (14), and
a flared section (20), extending from the conduit (30) and including the first open free end (13), the flared section (20) having an increasing diameter from the conduit (30) to the first open free end (13), and a part (21), referred to as top part, of the first open free end (13) being more radially spaced relative to the conduit (30) than another part (22), referred to as bottom part, of the first open free end (13), which is diametrically opposite the top part (21).

2. Device (10) according to claim 1, characterised in that the flared section (20) includes a first side (24), referred to as top side, which includes the top part (21) of the first open free end (13), a second side (23), referred to as bottom side, which includes the bottom part (22) of the first open free end (13), and in that the top side (24) of the flared section is flared relative to the conduit (30) according to an average angle (a) greater than the bottom side (23) of the flared section.

3. Device (10) according to claim 2, characterised in that the bottom side (23) of the flared section has a substantially straight profile.

4. Device (10) according to claim 2, characterised in that the bottom side of the flared section (20) is aligned relative to the conduit.

5. Device (10) according to claim 2, characterised in that the top side (24) of the flared section has a substantially “S”-shaped profile.

6. Device (10) according to claim 1, characterised in that the flared section (20) includes a lateral side, referred to as left lateral side, and another lateral side, referred to a right lateral side, which is diametrically opposite the left lateral side, and in that the left lateral side and the right lateral side are flared relative to the conduit (30) according to the same average angle.

7. Device (10) according to claim 1, characterised in that a geometric centre of the first open free end (13) is off-centred relative to a geometric centre of the conduit (30).

8. Device (10) according to claim 1, characterised in that it includes an insertion holding system (18).

9. Device (10) according to claim 8, characterised in that the insertion holding system (18) includes a bead formed at the outer periphery of the first open free end (13).

10. Device (10) according to claim 1, characterised in that it includes a positioning indicator (17).

11. Device (10) according to claim 10, characterised in that the positioning indicator (17) includes a rib which extends longitudinally along a part of a top side (16) of the device (10).

12. Device (10) according to claim 11, characterised in that the rib is configured to rigidify the collection device (10) in flexion.

13. Device (10) according to claim 11, characterised in that the rib extends longitudinally along at least a part of the top side of the conduit, or also along a part of the top side (24) of the flared section (20).

14. Device (10) according to claim 1, characterised in that the conduit (30) includes a connection endpiece (31) configured to connect a quantification receptacle thereto.

15. Device (10) according to claim 1, characterised in that the flared section (20) is made of a biocompatible material.

16. Device (10) according to claim 15, characterised in that the material has a hardness between 40 shore A and 80 shore A.

17. Device (10) according to claim 1, characterised in that the flared section (20) is configured to be inserted into a postpartum patient's vagina, for example in the vicinity of the outer orifice of the cervix.

18. Device (10) according to claim 17, characterised in that it is configured to form a vaginal device for collecting blood loss.

19. Device (10) according to claim 17, characterised in that the top part (21) is configured to be positioned in the patient's vagina towards the bladder whereas the bottom part (22) is configured to be positioned towards the rectum.

20. System for quantifying fluid loss including a device for collecting fluid loss (10) according to claim 1, and a quantification receptacle (40), ready to be connected to the conduit (30) of the device (10).
